(19) United States
(12) Patent Application Publication
(10) Pub. No.: US 2016/0310651 A1
(43) Pub. Date: Oct. 27, 2016

(54) ARTIFICIAL HEART

(71) Applicants: Enzo Tomasi, San Martino Buon Albergo (IT); Silvia Teresa Giustina Scuri, Milano (IT); Felice Mantovani, Tregnago (IT); Tiziano Perlato, Tregnago (IT); Gino Gerosa, Rovereto (IT); Iguzzini Illuminazione S.P.A., Padova (IT)

(72) Inventors: Silvia Teresa Giustina Scuri, Milano (IT); Felice Mantovani, Tregnago (IT); Tiziano Perlato, Tregnago (IT); Gino Gerosa, Rovereto (IT); Francesca Maria Susin, Padova (IT)

(21) Appl. No.: 15/104,929

(22) PCT Filed: Dec. 18, 2014
(86) PCT No.: PCT/IB2014/067068

§ 371 Date: Jun. 15, 2016

(30) Foreign Application Priority Data
Dec. 18, 2013 (IT) PD2013A000348

(51) Int. Cl. A61M 1/10; A61F 2/04; A61M 1/12

(52) U.S. Cl. CPC A61M 1/1037; A61M 1/1046; A61M 1/106; A61M 1/12; A61F 2/04

(57) ABSTRACT

An artificial heart (1) comprises two deformable lateral ventricles (11, 12) and a central ventricle (10), arranged in such a way that:—the central ventricle (10) is connected to the first lateral ventricle (11) and to the second lateral ventricle (12) by means of a first and a second one-way valve (21, 22), respectively, mounted so as to be able to be crossed by a hydraulic flow (B) from the first lateral ventricle (11) to the central ventricle (10) and then to the second lateral ventricle (12), respectively,—the first lateral ventricle (11) is connected to an inlet opening (31) and to the first one-way valve (21) and—the second lateral ventricle (12) is connected to the second one-way valve (22) and to a second outlet opening (32),—a mechanical and/or hydraulic actuator for alternately:—expanding the central ventricle (10) and compressing the lateral ventricles (11, 12).—compressing the central ventricle (10) and expanding the lateral ventricles (11, 12).

### CROSS REFERENCE TO RELATED APPLICATIONS

The present application claims priority to PCT International Application No. PCT/IB2014/067068 filed on Dec. 18, 2014, which application claims priority to Italian Patent Application No PD2013A000348 filed Dec. 18, 2013, the entirety of the disclosures of which are expressly incorporated herein by reference.

### STATEMENT RE: FEDERALLY SPONSORED RESEARCH/DEVELOPMENT

Not Applicable.

### FIELD OF THE INVENTION

The present invention relates to an artificial heart.

### BACKGROUND ART

With reference to the accompanying FIGS. 1, 2, 3 and 4, a plurality of types of known units for artificial hearts are indicated with reference numerals 1a, 2a, 3a and 4a, respectively. Such units are used in pairs to create an artificial heart, so as to reproduce the two halves of a biological heart, dedicated to the corporeal and systemic circulation, respectively.

The first type of known unit 1a for an artificial heart comprises a single compressible chamber having an inlet and an outlet opening for a blood flow. Each opening is provided with a respective one-way valve oriented so as to allow the inlet of the blood flow in the compressible chamber and the outlet of the blood flow from the compressible chamber, respectively. The first artificial heart 1a comprises mechanical and/or hydraulic means for alternately compressing and expanding the compressible chamber generating the ejection 1b and suction curves 1c, respectively, which represent the flow curves as a function of time through the outlet and inlet openings, respectively. The artificial heart 1a allows obtaining ejection and suction curves theoretically similar to those of a human heart. However, it has a series of embodiment drawbacks, the main of which is represented by the fact that in order to reduce the size of the unit it is necessary to increase the operating frequency. In this case, the flow rate pulse train generated deviates excessively from the operation of a natural heart. Consequently, the volume of the compressible chamber cannot be reduced beyond certain limits and this requires necessarily bulky devices, unsuitable in small-sized patients, such as women and teenagers.

The second type of known unit 2a is totally similar to the first heart 1a and allows realizing ejection 2b and suction curves 2c identical to the ejection 1b and suction curves 1c. In the second artificial heart 2a, the expansion and compression of the compressible chamber are carried out by means of an actuator active thereon. The second type of artificial heart 2a, representative of most of the devices that are or have been used in clinical use, has the same drawbacks described above with reference to the first type 1a.

The third type of known units 3a comprises two deformable ventricles arranged in series between an inlet opening, connected to the first ventricle, and an outlet opening, connected to the second ventricle. A partition wall translating with a periodic pattern is active between the two ventricles for alternately expanding the first ventricle and compressing the second ventricle or compressing the first ventricle and expanding the second ventricle. The translating partition wall is integral with a one-way valve which allows the passage of the blood flow from the first to the second ventricle. A second one-way valve is present at the outlet opening to allow the outflow of the blood flow from the second ventricle. The inlet opening is devoid of valves. The third artificial heart 3a allows obtaining, at each forward stroke of the translating partition wall, ejection 3b and suction curves 3c identical to each other. The return step to the initial position takes place in a predetermined time interval, corresponding to the horizontal stretches of the ejection 3b and suction curves 3c. The main drawback of the third artificial heart 3a is represented by the fact that the ejection and suction curves are generated only in the forward step of the translating partition wall. In fact, therefore, heart 3a requires a non-active operating step, only required to bring it to the initial conditions, without generation of flow. Furthermore, also in this case, the volume of the compressible chamber cannot be reduced beyond certain limits, for the same reasons described with reference to the first type 1a.

Such a drawback can be overcome with a fourth type of known units 4a, obtained by placing a first and a second deformable chamber, substantially identical to those of the second deformable heart 2a, alongside, A partition wall translating with a periodic pattern is active between the first and the second chamber for alternately expanding the first chamber and compressing the second chamber or compressing the first chamber and expanding the second chamber. The two chambers are mutually arranged so that the inlet and outlet openings of each chamber are opposite to those of the other chamber with respect to the translating partition wall. The inlet openings of the two chambers are connected to a common inlet conduit of the blood flow while the outlet openings are connected to a common outlet conduit. The configuration described above allows realizing the ejection 4b and suction curves 4c, identical to each other and are characterized by two peaks for each operating cycle, corresponding to the lower and upper dead center of the stroke of the translating partition wall, and a relative minimum point corresponding to the intermediate position between the upper and lower dead centers. In this case, in order to simulate the pulsating pattern of the human heart, the traversing partition wall is kept inactive for a predetermined time interval, corresponding to the horizontal stretches of the ejection 4b and suction curves 4c. The shape of the upper part of the ejection 4b and suction curves 4c allows obtaining a virtually identical flow rate, represented by curve 4d, reducing the size of the deformable chambers and at the same time increasing the frequency of the motion of the translating partition wall. In fact, by increasing the frequency, the pulse remains substantially identical, changing only the upper part of the flow curves. The main drawback of the fourth solution 4a is represented by the construction complexity which provides, for example, compared to the three previously described, four valves instead of two.

### SUMMARY OF THE INVENTION

The main object of the present invention is to provide an artificial heart which overcomes the drawbacks mentioned with reference to the cited prior art, allowing the same performance offered by the artificial heart of type 4a described above to be obtained, but with greater construction and operating simplicity.

Such an object is achieved by an artificial heart comprising a first deformable lateral ventricle, a deformable central ventricle and a second deformable lateral ventricle, said deformable ventricles being arranged in such a way that:
said central ventricle is connected to said first lateral ventricle and to said second lateral ventricle by means of a first one-way valve and a second one-way valve, respectively, said first and second one-way valves being mounted so as to be able to be crossed by a hydraulic flow from said first lateral ventricle to said central ventricle and from said central ventricle to said second lateral ventricle, respectively,
said first lateral ventricle is connected to an inlet opening of said hydraulic flow and to said first one-way valve and
said second lateral ventricle is connected to said second one-way valve and to a second outlet opening of said hydraulic flow,

said artificial heart further comprising a mechanical and/or hydraulic actuator for alternately:
expanding said central ventricle and compressing said first lateral ventricle and second lateral ventricle for generating a first passage of said hydraulic flow through said inlet and outlet openings, or
compressing said central ventricle and expanding said first lateral ventricle and second lateral ventricle for generating a second passage of said hydraulic flow through said inlet and outlet openings.

The artificial heart of the present invention allows obtaining the same suction and ejection curves obtainable with other known artificial hearts (in particular, the artificial heart 4a in FIG. 4) but with a simpler structure, in particular characterized by a minimum number of one-way valves.

Other advantages are obtained by means of an artificial heart implemented according to the dependent claims. In particular, the possibility to operate the valves independently of one another, synchronously or asynchronously. By suitably adjusting the de-phasing between the valves is possible to independently control each of the suction and ejection flow rates.

### BRIEF DESCRIPTION OF THE FIGURES

Further features and advantages of the invention will appear more clearly from the detailed description of preferred, but not exclusive, embodiments of an artificial heart according to the present invention, shown by way of a non-limiting example with the aid of the accompanying drawings, in which:

FIGS. 1-4 are four schematic sectional views with relevant operating diagrams of four respective known units for artificial heart;

FIG. 5 is a schematic sectional view with relevant operating diagram of a first embodiment variant of a unit for artificial heart according to the present invention;

FIGS. 6 and 7 are two schematic sectional views with relevant operating diagram of two respective embodiment variants of a unit for artificial heart according to the present invention;

FIGS. 8a-d are a plurality of schematic views of the unit in FIG. 5 in four respective operating steps;

FIGS. 9a-b, 10a-b and 11a-b are six schematic sectional views of six respective embodiment variants of a unit for artificial heart according to the present invention;

FIG. 12 is sectional view of the unit in FIG. 6.

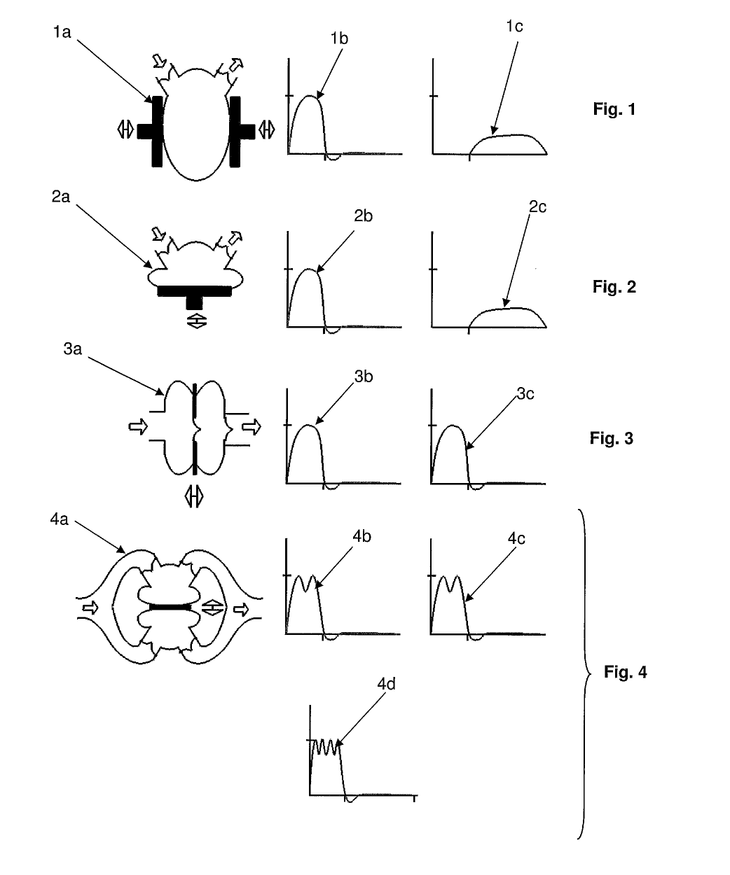

### DETAILED DESCRIPTION OF THE INVENTION

With reference to the accompanying figures, in all its possible embodiment variants, a unit for artificial heart, globally indicated with reference numeral 1, is usable for circulating a blood flow B in a human or animal body. The same unit 1 can be used for the pulmonary or systemic circulation. Normally, the use in pairs of two identical units 1, one dedicated to the pulmonary circulation, the other to the systemic circulation, allows replacing a biological, human or animal heart from the functional point of view. According to a possible embodiment variant of the present invention (not shown), a complete artificial heart consists of two units 1 coupled together in a single assembly.

Unit 1 comprises a first deformable lateral ventricle 11, a deformable central ventricle 10 and a second deformable lateral ventricle 12. The three deformable ventricles 10, 11, 12 are reciprocally arranged so that the central ventricle 10 is connected to the first lateral ventricle 11 and to the second lateral ventricle 12 by means of a first one-way valve 21 and a second one-way valve 22, respectively, which are mounted so as to be able to be crossed by the blood flow B from the first lateral ventricle 11 to the central ventricle 10 and from the central ventricle 10 to the second lateral ventricle 12, respectively.

The first lateral ventricle 11 is connected to an inlet opening 31 for the blood flow B and to the first one-way valve 21. The second lateral ventricle 12 is connected to the second one-way valve 22 and to a second outlet opening 32 of the blood flow B. Therefore, a path is formed in which the blood flow B crosses, in series, the inlet opening 31, the first lateral ventricle 11, the first one-way valve 21, the central ventricle 10, the second one-way valve 22, the second lateral ventricle 12 and finally the second outlet opening 32.

Unit 1 further comprises a mechanical and/or hydraulic actuator, better described hereafter for the different embodiment variants, for alternately:
expanding the central ventricle 10 and compressing said first lateral ventricle 11 and second lateral ventricle 12 so as to generate a first passage of blood flow B through the inlet and outlet openings 31, 32, or
compressing the central ventricle 10 and expanding the first lateral ventricle 11 and second lateral ventricle 12 so as to generate a second passage of blood flow B through the inlet and outlet openings 31, 32.

A plurality of possible embodiment variants of the present invention is described hereafter with reference to the accompanying figures. The different embodiment variants described, as well as those which may be obtained from by the appended claims, but not described in detail, are to be considered equivalent to each other. In the different figures, the same reference numerals therefore indicate the same or similar elements.

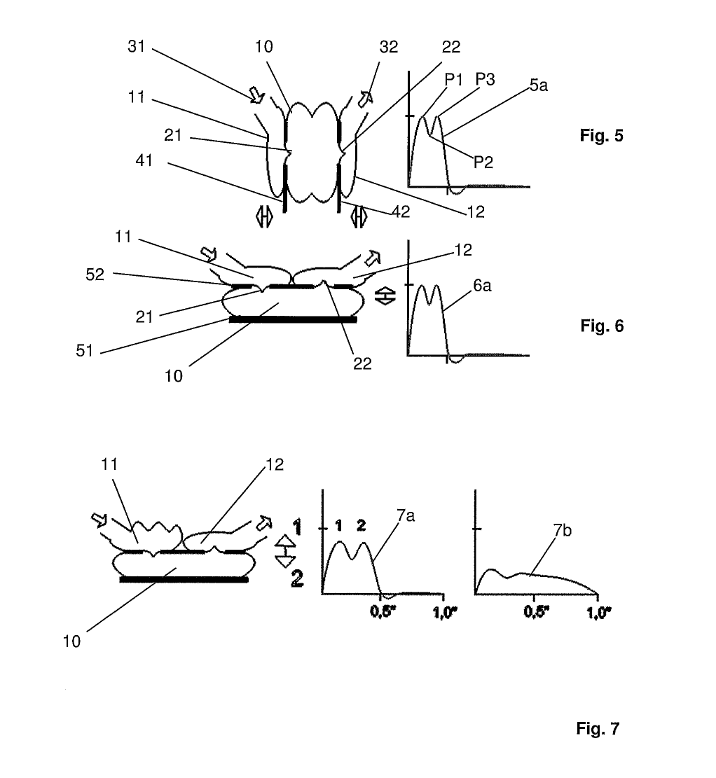

With initial reference to FIG. 5, the actuation of unit 1 according to the present invention is of the mechanical type and comprises:
a first movable partition wall 41 between the central ventricle 10 and the first lateral ventricle 11 to alternately compress or expand the central ventricle 10 and the first lateral ventricle 11,
a second movable partition wall 42, arranged parallel to the first movable partition wall 41, between the central ventricle 10 and the second lateral ventricle 12 to alternately compress or expand the central ventricle 10 and the second lateral ventricle 12.

In the embodiment variant in FIG. 5, the blood flow B crosses the unit for artificial heart 1 according to a predominantly linear pattern. The first and the second partition wall 41, 42 are movable in mutual spacing or approach for respectively expanding the central ventricle 10 and compressing the lateral ventricles 11, 12 or compressing the central ventricle 10 and expanding the lateral ventricles 11, 12. The first and the second one-way valve 21, 22 are integral to the first and second partition walls 41, 42, respectively, so that the motion of each partition walls 41, 42 in an opposing direction with respect to the blood flow B causes the opening of the valve integral thereto, respectively.

According to another embodiment variant (not shown), the two movable partition walls 41, 42 are mutually inclined and are movable fan-like so as to increase or decrease the mutual inclination for respectively expanding the central ventricle 10 and compressing the lateral ventricles 11, 12 or compressing the central ventricle 10 and expanding the lateral ventricles 11, 12.

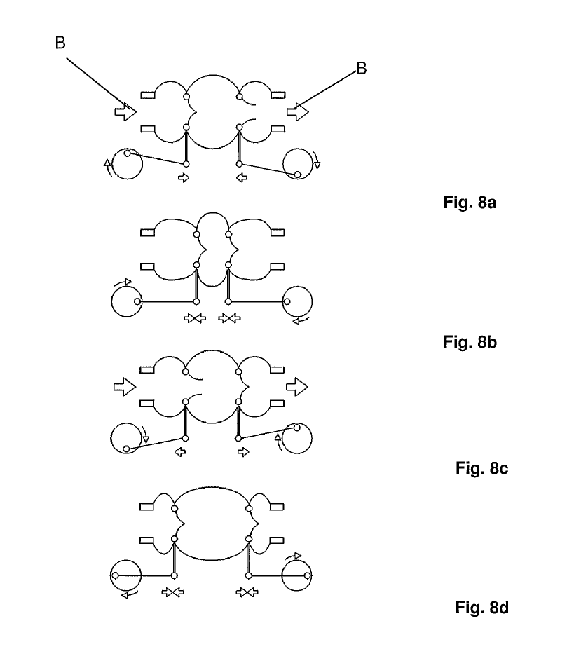

The operation of the embodiment variant in FIG. 5 is described hereafter in the various operating steps thereof with reference to the accompanying FIGS. 8a-d and to the diagram in FIG. 5a which shows both the suction flow curve through the inlet openings 31, and the ejection flow curve through the outlet openings 32.

From a neutral position (FIG. 8a) in which the central and lateral ventricles 10, 11, 12 are in an undeformed configuration, partition walls 41, 42 are activated in mutual approach by means of a thrust crank mechanism or other mechanical system per se known and conventional, able to move partition walls 41, 42 while maintaining the mutual parallelism. By approaching each other, partition walls 41, 42 compress the central ventricle 10 and expand the lateral ventricles 11, 12 up to complete the first quarter of total stroke in which they reach the first dead center, corresponding to the position of minimum distance between partition walls 41, 42 (FIG. 8b) and to the first maximum point P1 of the flow curve 5a. During the approach stroke of partition walls 41, 42, the first valve 21 is closed while the second valve 22 is open. In the first dead center, both valves 21, 22 are closed. During the stroke of mutual approach of the partition walls, the expansion of the first lateral ventricle 11 with first one-way valve 21 closed allows the suction of the blood flow B through the inlet opening 31, At the same time, the compression of the central ventricle 10 and the opening of the second one-way valve 22 allow the ejection of the blood flow B through the outlet opening 32. During the approach stroke of the partition walls, a first passage of blood flow B through the inlet and outlet openings 31, 32 is therefore generated.

The stroke of mutual spacing of partition walls 41, 42 starts from the position of first dead center, during which the first valve 21 opens while the second valve 22 remains closed. During the spacing stroke, the central and lateral ventricles 10, 11, 12 return to the undeformed configuration (FIG. 8c) completing the second quarter of total stroke, corresponding to the relative minimum point P2 of the flow curve 5a, Continuing the mutual spacing stroke, partition walls 41, 42 compress the lateral ventricles 11, 12 and expand the central ventricle 10 up to complete the third quarter of total stroke in which they reach the second dead center, corresponding to the position of maximum distance between partition walls 41, 42 (FIG. 8d) and to the second maximum point P3 of the flow curve 5a. Also in the second dead center, both valves 21, 22 are closed. During the stroke of mutual spacing of partition walls 41, 42, the expansion of the central ventricle 10 and the opening of the first one-way valve 21 allow the suction of the blood flow B through the inlet opening 31. At the same time, the compression of the second lateral ventricle 12 and the closing of the second one-way valve 22 allows the ejection of the blood flow B through the outlet opening 32. During the mutual spacing stroke of partition walls 41, 42, a second passage of blood flow B through the inlet and outlet openings 31, 32 is therefore generated.

The mutual approach stroke of partition walls 41, 42 starts from the position of second dead center, during which the second valve 22 opens while the first valve 21 remains closed and which corresponds to the fourth quarter of total stroke, at the end of which unit 1 returns to the undeformed configuration in FIG. 8a.

The flow curve 5a has a shape virtually identical to that of the flow curves 4b, 4c of the fourth known type of unit for artificial heart 4a. Therefore, by decreasing the size of ventricles 10, 11, 12 and increasing the actuating frequency of partition walls 41, 42 it is possible to obtain flow curves identical to curve 4d of the fourth known type of unit for artificial heart 4a.

Alternatively, according to another operating mode of the embodiment variant in FIG. 5, partition walls 41, 42 are actuated asynchronously in order to reach the points of first and second dead center at different times, so as to generate ejection and suction curves different from each other. By adjusting the de-phasing between the motions of partition walls 41, 42 it is possible to suitably adjust the flow rate and the shape of the ejection curve.

With reference to a second embodiment variant (FIG. 6), the blood flow B crosses unit 1 according to a mainly “U” shaped pattern, in which the first descending arm consists of the first ventricle 11, the horizontal stretch of the central ventricle 10 and the second ascending arm of the second ventricle 12. In this variant, the actuation is still of the mechanical type and comprises:
a base 51 integral on the exterior with the central ventricle 10,
a movable partition wall 52 parallel to base 51 and interposed between the central ventricle 10 and the first and second lateral ventricles 11, 12, the first and the second one-way valve 21, 22 being both integral with partition wall 52.

Base 51 and partition wall 52 are movable in mutual spacing or approach in a way similar to the first and the second partition wall 41, 43 of the first embodiment variant in FIG. 5, so as to form suction and ejection curves 6a equal to curve 5a. In the case of mutual spacing, base 51 and partition wall 52 expand the central ventricle 10 and compress the first lateral ventricle 11 and the second lateral ventricle 12. In the case of mutual approach, base 51 and partition wall 52 compress the central ventricle 10 and expand the first lateral ventricle 11 and the second lateral ventricle 12.

The parallelism between base 51 and partition wall 52 allows maintaining the distance between the first one-way valve 21 and base 51 equal to the distance between the second one-way valve 22 and base 51 so as to synchronize the passage of the blood flow B through the first and the second valve 21, 22.

Alternatively, base 51 and partition wall 52 are mutually inclined so that the distance between the first valve 21 and base 51 is different from the distance between the second valve 22 and base Si. In this way, similarly to what is achievable in the first embodiment variant by de-phase the motion of partition walls 41, 42, it is therefore possible to suitably de-phase the passage of the blood flow B through the first and the second valve 21, 22, obtaining ejection and suction curves different from each other.

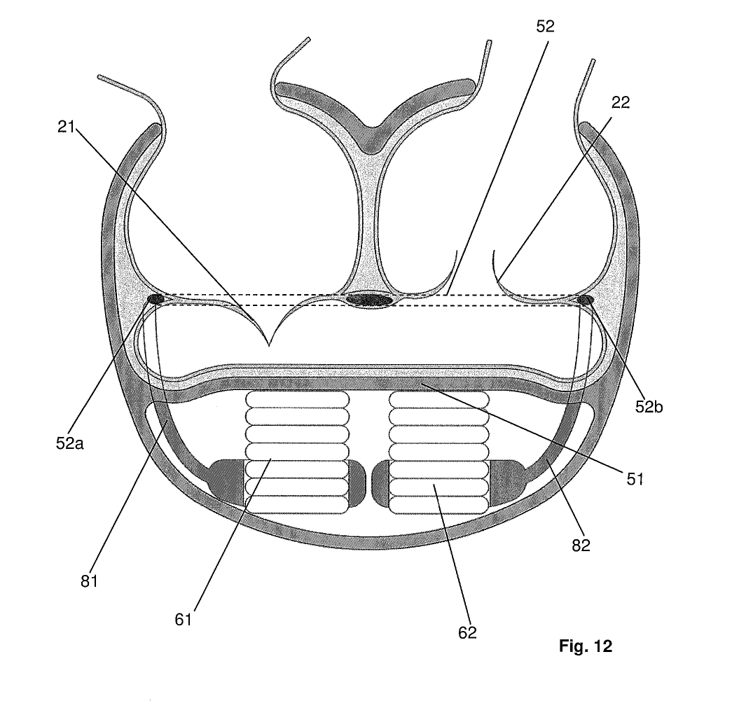

In general, according to the present invention, the actuating means of ventricles 10, 11, 12 placed at the first valve 21, may be made independent of the actuating means placed at the second valve 22 so as to obtain in each case ejection and suction curves different from each other and adjustable. To this end, with reference to FIG. 12 which shows a solution with blood flow in the shape of “U” like that in FIG. 6, it is possible to use two linear motors 61, 62 located externally to base 51 of the central ventricle 10 and connected, by means of two connecting members 81, 82, to two opposite ends 52a,b of partition wall 52, respectively, arranged in the vicinity of the one-way valves 21, 22, respectively. By moving the two opposite ends 52a,b independently of each other, it is possible to obtain the desired inclination between base 51 and partition wall 52. In the example in FIG. 12, moreover, such an inclination is adjustable.

According to another embodiment variant of the present invention (not shown), each of the one-way valves 21, 22 is moved by a respective actuator completely independent of the other, obtained for example by dividing partition wall 52 in FIG. 6 in two partition walls active on one and the other of the one-way valves 21, 22, respectively. In the latter variant the advantage is obtained that if one of the two actuators stops running, thus stopping one of the one-way valves 21, 22, with reference to curve 5a, the pulsation is reduced to only one peak (for example peak P1) per cycle, thus halving the flow rate. In this condition, it is therefore sufficient to increase the frequency thus obtaining a minimum flow to ensure survival.

With reference to the embodiment variant in FIG. 7, the first lateral ventricle 11 has a greater deformability compared to the second lateral ventricle 12 and with respect to the central ventricle 10. This allows obtaining ejection and suction curves 7a, 7b different from each other, with a suction capacity of non-zero even when the ejection flow rate is null, as happens for the first and the second type of known unit for artificial heart 1a, 2a described above. Moreover, in this latter variant, the greater deformability of the first lateral ventricle 11 allows it to also act as a compensation and accumulation organ.

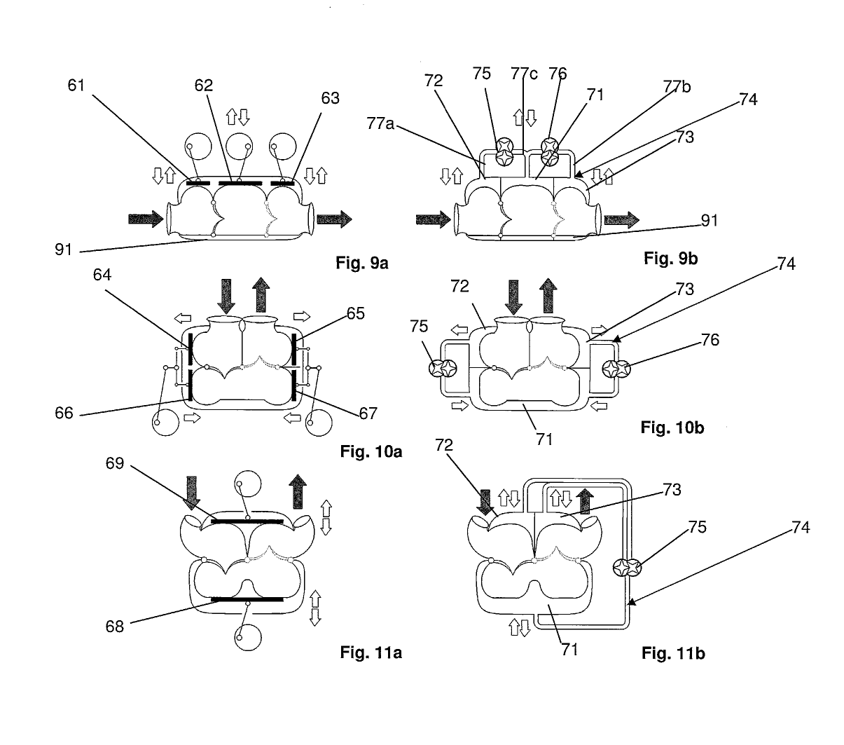

With reference to FIGS. 9a-b, 10a-b and 11a-b, it is possible to obtain embodiment variants of unit 1 in which the first and the second valve 21, 22 are fixed and the mechanical or hydraulic actuation is active to compress or expand the central ventricle 10 and the lateral ventricles 11, 12.

With reference to FIGS. 9a, 10a, 11a, the actuation is mechanical and comprises a plurality of walls integral on the exterior with the central ventricle 10 and the lateral ventricles 11, 12. In general, for such embodiment variants, the walls are movable in a coordinated manner for alternately:
expanding the central ventricle 10 and compressing said first lateral ventricle 11 and second lateral ventricle 12 so as to generate a first passage of blood flow B through the inlet and outlet openings 31, 32, or
compressing the central ventricle 10 and expanding the first lateral ventricle 11 and second lateral ventricle 12 so as to generate a second passage of blood flow B through the inlet and outlet openings 31, 32.

The configuration in FIG. 9a is of the linear type and comprises a rigid base 91, common to all the central and lateral ventricles 10, 11, 12, and three walls 61, 62, 63, integral on the exterior with the first lateral ventricle 11, the central ventricle 10 and the second lateral ventricle 12, respectively. Walls 61, 62, 63 are arranged parallel to the rigid base 91, so that ventricles 10, 11, 12 are interposed between the respective wall and the rigid base 91. Walls 61, 62, 63 are connected to moving means per se known and conventional, respectively, for moving walls 61, 62, 63 in a coordinated manner as described above.

The configuration in FIG. 10a is of the “U” shaped type and comprises:
a first pair of walls 64, 65 arranged in parallel along the descending stretch of the “U” and integral on the exterior with the first lateral ventricle 11 and the central ventricle 10, respectively,
a second pair of walls 66, 67 is further provided, arranged in parallel along the ascending stretch of the “U” and integral on the exterior with the second lateral ventricle 12 and the central ventricle 10, respectively.

The two pairs of walls 64, 65 and 66, 67 are arranged with respect to ventricles 10, 11, 12 in such a way that the two lateral ventricles 11, 12, arranged side by side, are interposed between one of the walls of the first pair of walls 64, 65 and one of the walls of the second pair of walls 66, 67 and that the central ventricle is interposed between the other walls of the two pairs of walls 64, 65 and 66, 67.

Each pair of walls 64, 65 and 66, 67 is connected to a respective moving member per se known and conventional to respectively move them in a coordinated manner, as described above.

The configuration in FIG. 11a is of the “U” shaped type and comprises a first wall 68 integral on the exterior with the central ventricle 10 and arranged in parallel along the horizontal stretch of the “U” and a second wall 69, parallel to the first wall 68 and integral on the exterior with both lateral ventricles 11, 12. The two walls 68 and 69 are arranged in such a way that the assembly of ventricles 10, 11, 12 is interposed between them. Walls 68 and 69 are connected to respective moving members per se known and conventional to respectively move them in a coordinated manner, as described above.

With reference to FIGS. 9b, 10b, 11b, the actuation is of the hydraulic type and comprises:
a first hydraulic chamber 71 adjacent to the central ventricle 10, a second hydraulic chamber 72 and a third hydraulic chamber 73, adjacent to the lateral ventricles 11, 12. respectively,
a hydraulic circuit 74 for drawing or delivering a service fluid from or to chambers 71, 72, 73 in a coordinated manner for alternately:
expanding the central ventricle 10 and compressing the first lateral ventricle 11 and the second lateral ventricle 12 so as to generate a first passage of blood flow B through the inlet and outlet openings 31, 32, or
compressing the central ventricle 10 and expanding the first lateral ventricle 11 and second lateral ventricle 12 so as to generate a second passage of blood flow B through the inlet and outlet openings 31, 32.

The configuration in FIG. 9b is of the linear type and comprises a rigid base 91, common to all central and lateral ventricles 10, 11, 12. Chambers 71, 72, 73 are arranged parallel to the rigid base 91, so that ventricles 10, 11, 12 are interposed between the respective chamber and the rigid base 91. The hydraulic circuit 74 comprises two hydraulic pumps 75, 76, a first branch 77a of connection between the first hydraulic pump 75 and the second hydraulic chamber 72, a second branch 77b of connection between the second hydraulic pump 76 and the third hydraulic chamber 73, and a third branch Y-shaped 77c of connection between both hydraulic pumps 75, 76 and the first hydraulic chamber 71. The two hydraulic pumps 75, 76, for example of the lobed type or other conventional type, are actuated so as to draw or deliver a service fluid from or to chambers 71, 72, 73 in a coordinated manner. as described above.

In the configuration in FIG. 10b, of the “U” shaped type, the first hydraulic chamber 71 is arranged around the central ventricle 10, on the exterior thereof, the second hydraulic chamber 72 is alongside the first lateral ventricle 11, along the descending stretch of the “U” and the third hydraulic chamber 73 is alongside the second lateral ventricle 12, along the ascending stretch of the “U”. The hydraulic circuit 74 comprises two hydraulic pumps 75, 76 interposed between the first and the second chamber 71, 72 and the first and the third chamber 71, 73. respectively. The two hydraulic pumps 75, 76 are actuated so as to draw or deliver a service fluid from or to chambers 71, 72, 73 in a coordinated manner, as described above.

The configuration in FIG. 11b is also of the “U” shaped type and differs from that in FIG. 10b substantially in that it comprises a single hydraulic pump 75, for example of the lobed type or other conventional type, having a first suction or delivery side connected to the first hydraulic chamber 71, and the other side connected to both the second and the third chamber 72, 73. In this way, the rotation of pump 75 in one direction of rotation expands the central ventricle 10 and compresses the lateral ventricles 1, 12 while the rotation in the opposite direction compresses the central ventricle 10 and expands the lateral ventricles 11, 12.

In general, in all possible embodiment variants, the unit for the artificial heart of the present invention allows generating the required flow rate limiting to the minimum the number of valves (two), allowing the implementation of a simpler and more reliable structure than what proposed by the prior art.

1. An artificial heart comprising a first deformable lateral ventricle a central ventricle and a second deformable lateral ventricle said deformable ventricles being arranged in such a way that:
said central ventricle is connected to said first lateral ventricle and to said second lateral ventricle by means of a first one-way valve and a second one-way valve respectively, said first and second one-way valves being mounted so as to be able to be crossed by a hydraulic flow from said first lateral ventricle to said central ventricle and from said central ventricle to said second lateral ventricle respectively,
said first lateral ventricle is connected to an inlet opening of said hydraulic flow and to said first one-way valve and
said second lateral ventricle is connected to said second one-way valve and to a second outlet opening of said hydraulic flow,
said artificial heart further comprising a mechanical and/or hydraulic actuator for alternately:
expanding said central ventricle and compressing said first lateral ventricle and second lateral ventricle for generating a first passage of, said hydraulic flow through said inlet and outlet openings, or
compressing said central ventricle and expanding said first lateral ventricle and second lateral ventricle for generating a second passage of said hydraulic flow through said inlet and outlet openings.

2. The artificial heart according to claim 1, wherein said mechanical actuator comprises:
a first movable partition wall between said central ventricle and said first lateral ventricle to alternately compress or expand said central ventricle and said first lateral ventricle,
a second movable partition wall between said central ventricle and said second lateral ventricle to alternately compress or expand said central ventricle and said second lateral ventricle,
said first and second partition walls being movable in mutual spacing or approach tor expanding said central ventricle and compressing said first lateral ventricle and second lateral ventricle or compressing said central ventricle and expanding said first lateral ventricle and second lateral ventricle, respectively, said first and second one-way valves being integral to said first and second partition walls, respectively.

3. The artificial heart according to claim 1, wherein said mechanical actuator comprises:
a base integral on the exterior with said central ventricle,
a movable partition wall interposed between said central ventricle and said first and second lateral ventricles,
said first and second partition walls being movable in mutual spacing or approach for expanding said central ventricle and compressing said first lateral and second lateral ventricle or compressing said central ventricle and expanding said first lateral ventricle and second lateral ventricle, respectively, said first and second one-way valves being both integral to said partition wall.

4. The artificial heart according to claim 3, wherein said, base and partition wall are parallel to each other so as to maintain the distance between said first one-way valve and said base equal to the distance between said second one-way valve and said base so as to synchronize the passage of said hydraulic flow through said first and second one-way valves.

5. The artificial heart according to claim 3, wherein said base and partition wall are mutually inclined so as to maintain the distance between said first one-way valve and said base different from the distance between said second one-way valve and said base so as to de-phase the passage of said hydraulic flow through said first and second one-way valves.

6. The artificial heart according to claim 1, wherein said first and second one-way valves are fixed and said mechanical actuator comprises a plurality of walls integral on the exterior with said central ventricle and said first lateral ventricle and second lateral ventricle, said plurality walls being movable in a coordinated manner for alternately:
expanding said central ventricle and compressing said first lateral ventricle, or
compressing said central ventricle and expanding said first lateral ventricle and second lateral ventricle for generating a second passage of said hydraulic flow through said inlet and outlet openings.

7. An The artificial heart according to claim 1, wherein said first and second one-way valves are fixed and said hydraulic actuator includes:
at least three hydraulic chambers adjacent to said central ventricle and to said first lateral ventricle and second lateral ventricle, respectively,
a hydraulic circuit for drawing or delivering a service fluid from or to said chambers in a coordinated manner for alternately:
expanding said central ventricle and compressing said first lateral ventricle and second lateral ventricle for generating a first passage of said hydraulic flow through said inlet and outlet openings, or
compressing said central ventricle and expanding said first lateral ventricle and second lateral ventricle for generating a second passage of said hydraulic flow through said inlet and outlet openings.

8. The artificial heart according to claim 1, wherein one of said lateral ventricles has a lower deformability than the other of said lateral ventricles, thus acting as compensation and accumulation member for said hydraulic flow.
